(19) United States
(12) Patent Application Publication
(10) Pub. No.: US 2008/0249600 A1
(43) Pub. Date: Oct. 9, 2008

(54) STENTS WITH DRUG RESERVOIR LAYER AND METHODS OF MAKING AND USING THE SAME

(75) Inventors: Liliana Atanasoska, Edina, MN (US); Jan Weber, Maastricht (NL); Robert W. Warner, Woodbury, MN (US)

Correspondence Address: MERCHANT & GOULD PC, P.O. BOX 2903, MINNEAPOLIS, MN, 55402-0903, US

(73) Assignee: Boston Scientific Scimed, Inc., Maple Grove, MN (US)

(21) Appl. No.: 11/697,401

(22) Filed: Apr. 6, 2007

(51) Int. Cl. A61F 2/82

(52) U.S. Cl. 623/1.4; 623/1.42

(57) ABSTRACT

A method of making drug eluting stents comprises forming ceramic surface coatings of two or more levels of porosity on a stent body. The less porous coating is more conducive to endothelial cell growth, while the more porous coating is better suited for storing and releasing drugs. The surface coatings of different porosities can be produced by coating stent body surface of differing roughness with coatings made by sol-gel method. Differing roughness of the stent body surface can be produced by selective etching of the stent body surface.

### TECHNICAL FIELD

This disclosure relates to stents and related methods. Specific arrangements also relate to methods and configurations of stents with drug reservoir layers.

### BACKGROUND

Stents are prosthetic devices typically intraluminally placed by a catheter within a vein, artery, or other tubular body organ for treating conditions such as, occlusions, stenoses, aneurysms, dissection, or weakened, diseased, or abnormally dilated vessel or vessel wall, by expanding the vessel or by reinforcing the vessel wall. Stents can improve angioplasty results by preventing elastic recoil and remodeling of the vessel wall and treating dissections in blood vessel walls caused by balloon angioplasty of coronary arteries.

Stents are typically tubular and expandable from a collapsed state to an expanded state. In a typical operation to implant a stent, the stent is initially configured in the collapsed state, with a cross-sectional size sufficiently small for ease of passage to the intended site. After the stent reaches the intended site, the stent is typically deformed to increase its cross-sectional size to fully engage the stent with the surrounding tissues. The stent thereafter remains in place in the expanded state.

In some cases, stents are loaded with drugs to be released over time to treat various conditions. Drugs are typically dispersed in coating layers on the surfaces of stents.

While conventional stent technology is relatively well developed, technologies related to drug-delivering stents are still being developed.

### SUMMARY OF THE DISCLOSURE

The present disclosure relates generally to a method of making drug reservoir surface coating layers on stent bodies. In one arrangement, the method includes forming a drug containing ceramic film using sol-gel methods.

A further aspect of the present disclosure relates to stents with drug reservoir surface coating layers. In one configuration, stents have surface portions that are configured to be conducive to cell growth on the stent. The stents further have other surface portions that are configured to be conducive to storing drugs and releasing the drugs at desired rates.

### BRIEF DESCRIPTION OF THE DRAWINGS

FIG. 1 is a schematic perspective view of an example stent constructed according to on aspect of the present disclosure.

FIG. 2 is a schematic cross-sectional view of a portion of the stent shown in FIG. 1.

FIG. 3 is a schematic diagram showing the formation of a drug-polymer complex according to one aspect of the present disclosure.

### DETAILED DESCRIPTION

### I. Overview

This disclosure relates to making stents having surface layers, with portions of the surface layers configured as drug reservoir layers, and other portions configured to promote endothelial cell growth, thereby enhancing the integration of stents into their surrounding tissues.

Stents are typically implanted intraluminally in tubular body organs, such as blood vessels, to expand or strengthen the portion of the organ where the stent is placed. It is typically desirable for a stent to have surface portions capable of promoting cell growth over the surfaces of the stent. For drug eluting stents, it is also desirable for a stent to have surface portions capable of storing drugs and releasing the drugs at particular rates. Generally, a set of surface characteristics that are best suited for cell growth may not be best suited for a drug reservoir. For example, a surface layer with a highly porous structure is typically well suited for drug reservoir applications. However, such a structure may not be ideal for cell growth.

One solution according to an aspect of the present disclosure is to provide a stent comprising a stent body with ceramic coating layer having portions of two different porosities. Portions of the stent body surface are etched, e.g., by chemical or laser etching to produce a greater surface roughness than other portions of the stent body. In one aspect of the present disclosure, at least 10% of the nominal total stent body surface area is etched to produce a greater surface roughness than at least another 10% of the nominal total stent body surface area. The nominal total stent body surface area is measured at scales sufficiently greater than the sizes of the pores such that surface roughness due to porosity can be ignored. In other aspects of the disclosure, at least 20% or 30% of the nominal total stent body surface area is etched. The difference in roughness between a first, etched portion and a second portion is generally at least sufficient to enable significant clinically different applications for the two portions, respectively. In one aspect of the present disclosure, the difference is such that the a ceramic layer formed on the first portion is a more suitable drug reservoir layer; and a ceramic layer formed on the second portion is a more suitable substrate for cell growth. According to one aspect of the present disclosure, the first, etched portion of the stent body surface has a higher porosity than the second portion by at least about 50%. The porosity here is measured in volume fraction occupied by the pores. In other aspects of the disclosure the first portion has a higher porosity than the second portion by at least 100%, 500% and 1000%, respectively. A sol-gel process is then used to form ceramic coating layers over the stent body surface portions. The ceramic coating layers over the etched portions of the stent body surface possess a relatively higher porosity (e.g., by at least about 50%, 100% or 500%) than the ceramic coating layers over the non-etched portions of the stent body.

In a further aspect of the present disclosure, portions of a stent, e.g., the abluminal (i.e., away from the lumen, or toward vessel wall) surface portions and at least a portion of the lateral surfaces, are configured as reservoirs to store drugs, such as antirestenotics, antibiotics and anti-proliferative drugs, and to locally release the drugs over time at predetermined rates. Other portions of the stent, e.g., the adluminal (i.e., toward the lumen) surface portions, are configured to promote endothelial cell growth on the stent. In one example drug-eluting stent, the adluminal surface of the stent comprises drug reservoir portions having a porous ceramic layer; the abluminal surface of the stent comprises portions with smooth ceramic coating conducive to endothelial cell growth.

### II. Example Configurations

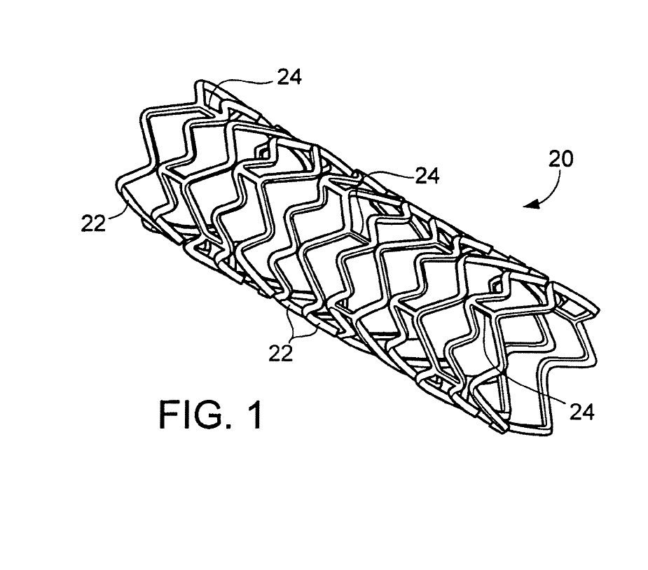

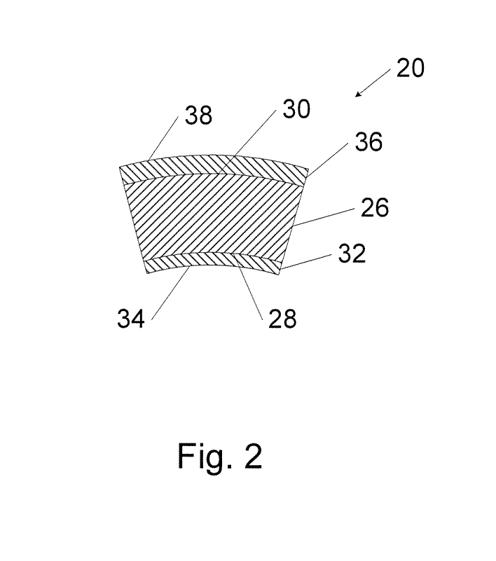

A process for making a stent with drug reservoir coating layers is now described with reference to an example stent 20 in FIGS. 1 and 2. The stent 20 is shown in an expanded state. The stent 20 has the form of a tubular member defined by a plurality of bands 22 and a plurality of connectors 24 that extend between and connect adjacent bands. During use, bands 22 are expanded from an initial, smaller cross-sectional size to a larger one to contact stent 20 against a wall of a vessel, thereby expanding or strengthening the vessel. A stent can be expanded using a variety of methods. For example, one or more balloons can be used to expand a stent. A self-expanding stent can also be compressed into a collapsed state and held in the collapsed state by a sheath prior to implantation, and unsheathed and permitted to expand at the implantation site. Examples of self-expanding stents include stents made of memory metals, which are flexible and collapsible from a predefined shape at room temperature but regains the predefined shape above certain critical temperature. Connectors 24 provide stent 20 with flexibility and conformability so that the stent can adapt to the contours of the vessel.

As schematically shown in FIG. 2, the stent 20 comprises a stent body 26. In accordance with one aspect of the present disclosure, a method for making a stent 20 comprises making surface portions of the stent body 26 rougher than other surface portions and subsequently forming ceramic layers over the stent body surface portions of differing roughness to produce different porosities in the ceramic layers. Surface portions of the stent body 26 can be processed, for example, by chemical etching or laser etching, to produce a greater roughness than other surface portions. Sol-gel processes can be used to form ceramic layers that have greater porosity over the roughened surface portions of the stent body 26 than over other surface portions.

As an example, as schematically shown in FIG. 2, the stent body 26 comprises an adluminal surface 28 and abluminal surface 30. The abluminal surface 30 is etched to result in a roughness that is significantly greater than the roughness of the adluminal surface 28. An adluminal ceramic layer 32 is coated on the adluminal surface 28. An abluminal ceramic layer 36 is coated on the abluminal surface 30. Both ceramic layers 32, 36 are formed by a sol-gel process. As a result, the adluminal ceramic layer 32 is a densely packed layer, which is conducive to cell growth; and the abluminal ceramic layer 36 is a porous layer, which can serve as a drug reservoir.

In one aspect of the present disclosure, the porosity of the etched surface portions range from about 15% to about 90%, and the porosity of the unetched surface portions range from about 0% to about 10%. The porosities in this example are expressed in terms of volume fractions occupied by the pores. Various techniques for measuring porosity are known. Examples include helium pycnometry, mercury porosimetry, physical gas adsorption techniques, Rutherford backscattering spectroscopy (RBS) and cyclic voltammetry (CV). Specific examples of porosity measurement techniques include: P. Klobes et al., Porosity and Specific Surface Area Measurement for Solid Materials, NIST Recommended Practice Guide, NIST SP 960-17 (2006); M. Lakatos-Varsanyi et al, Cyclic voltammetry measurements of different single-, bi- and multilayer TiN and single layer CrN coatings on low-carbon-steel substrates, Corrosion Science, Volume 41, Number 8, 1 Aug. 1999, pp. 1585-1598; H. A. Ponte et al, Porosity determination of nickel coatings on copper by anodic voltammetry, Journal of Applied Electrochemistry, Volume 32, Number 6, pp. 641-646 (2002); and V. Torres-Costa et al, RBS characterization of porous silicon multilayer interference filters, Electrochemical and Solid-State Letters, 7(11), G244-G246 (2004). The above-cited references are incorporated herein by reference.

A. Surface Treatment of Stent Body

As mentioned above, the textures of the adluminal surface 28 and abluminal surface 30 can be controlled by appropriate surface processing. In one aspect of the present disclosure, the greater roughness of the abluminal surface 30, as compared to that of the adluminal surface 28, is produced by etching the abluminal surface 30. Various metal etching techniques are known and can be used. For example, chemical etching and laser etching methods can be used in the alternative or in combination. For chemically etching stainless steel, for example, a mixture of nitric acid and hydro fluoric acid can be used. Examples of usable laser etching techniques can be found in J. Philip et al., “Laser based etching technique for metallography and ceramography”, Materials Science and Engineering A, Vol. 338, 1-2 (2002), pp. 12-23, and A. Kruusing, “Underwater and water-assisted laser processing: Part 2—Etching, cutting and rarely used methods”, Optics and Lasers in Engineering, 41 (2004), pp. 329-352. Both references above are incorporated herein by reference.

To create surfaces of different roughness with chemical etching, etching resists can be used to cover up the surface portions where etching is not desired. For example, a negative photo-chemical resist can be applied to the stent body 26, and the portions of the resist are irradiated with, for example, ultraviolet light. The irradiated resist is then washed with a developer to remove the un-irradiated portions, thereby exposing the corresponding portions of the stent body surface underneath. A photo-chemical resist mask is thus formed, protecting the portions of the stent body 26 covered by the photo-chemical resist from etching. The masked stent body 26 is then subjected to the etching chemicals to etch the portions unprotected by the mask. Suitable photo-chemical etching resists can be obtained from variety of sources, including HTP HiTech Photopolymere AG, Basel, Switzerland.

B. Formation of Ceramic Surface Coating Layers

According to an aspect of the present disclosure, the ceramic layers 32, 36 are formed by a sol-gel process. The process is based on complex formation between (a) templating agents such as Pluronic F127 nonionic block-copolymer and polyethylene glycol (PEG), (b) hydrolysis/condensation inhibitors such as carboxylic acids, keto-esters and amines, and (c) partially hydrolyzed titanium alkoxides. Various examples of such processes are known and can be used. For example, a procedure for forming an oxide film on metal substrate is disclosed in S. V. Lamaka et al., “TiOₓ self-assembled networks prepared by templating approach as nanostructured reservoirs for self-healing anticorrosion pre-treatments”, Electrochemistry Communications, 8 (2006), pp. 421-428, which is incorporated herein by reference. A TiOₓ based sol is prepared at room temperature by hydrolysis of tetraisopropyl orthotitanate (Ti(OCH(CH₃)₂)₄ or Ti(OiPr)₄). Ti(OiPr)₄ is added to ethanol solution of Pluronic F127 in 1:30 weight ratio and stirred for 1 hour to form a precursor. The precursor is then hydrolyzed by addition of acidified water (pH 1) in 1:100 precursor-to-water molar ratio. Both the adluminal and abluminal sides of the stent body 26 is then coated with the resultant sol. In one example, coating is accomplished by dipping the stent body 26 in the sol for a period of time (for example, 3 minutes), and then withdrawing the stent body 26 from the sol at a controlled speed (for example, 18 cm/min). The assembly of the stent body 26 coated with a TiOₓ based sol is then heated (for example, at 250° C.) to above supercritical temperature of the sol to dry the coating to form a layer of titanium oxide film, which substantially replicates the porous structure of the stent body and is believed to bear an epitaxial relationship with the stent body.

The materials and processing parameters can be varied to achieve the desired properties of the ceramic coatings. For example, combinations of hydrostatic pressure and solvent type can be chosen to achieve a desired solubility of the metal salts (e.g., Ti(OiPr)₄) in the sol and to vary the supercritical temperature of the sol to achieve desired drying temperatures for the coating. For example, in certain applications, as set forth in more detail below, drugs can be incorporated into the sol during the sol-gel process to impregnate the final ceramic coating with the drugs. High-temperature drying may not be desirable in such applications as it may drive off the drugs from the coating. By using appropriate solvents and/or pressure for dissolving the metal salts, the coating can be dried at desirably low temperatures, for example 200° C. or lower. For example, various solvothermal sol-gel processes can be used for low-temperature synthesis of drug eluting ceramic coating layers in stents.

Further examples of sol-gel processing for coating substrates are disclosed in J. C. Yu et al, “Enhanced photocatalytic activity of mesoporous and ordinary TiO₂ thin films by sulfuric acid treatment”, Applied Catalysis B: Environmental 36 (2002) 31-43; and A. Chougnet et al., “Substrates do influence the ordering of mesoporous thin films”, J. Mater. Chem., 15 (2005), 3340-3345. All references above are incorporated herein by reference.

The process outlined above is capable of producing ceramic coating on the order of a few micrometers thick or thicker. Further, the process can result in different surface morphologies for different surface portions of the same stent. In one example, the titanium oxide layer 32 on the smooth adluminal surface 28 of the stent body 26 has a uniform thickness with densely packed titanium oxide particles having a narrow size distribution. In contrast, the titanium oxide layer 36 on the etched abluminal surface 30 of the stent body 26 has a highly porous, network-like structure. The resultant relatively smooth adluminal surface 34 of the uniform layer 32 is conducive to endothelial cell grown thereon. In contrast, the porous abluminal surface layer 36 has a high surface area and is thus suitable for storing drugs.

C. Materials Used

Various materials can be used to make stents with ceramic coating layers with portions of differing porosity. The stent body 26, for example, can be made of a variety of materials that are known, or later found, to be suitable for endoluminal implantation applications. For example, a variety of metals that have requisite mechanical properties (such as strength and deformability) are biocompatible and suitable as substrates for forming ceramic coatings can be used. Such metals include various alloys and other metals. In one configuration, the stent body 26 is made of stainless steel.

The ceramic layers, such as those labeled 32 and 36 can be made of a variety of ceramic materials that are biocompatible and compatible with the drug or drugs intended to be stored in the ceramic layers. Ceramic materials are solids that have as their essential component, and are composed in large part of, inorganic nonmetallic materials. Examples of suitable ceramic materials include certain transition metal oxides, such as titanium (TiOₓ), tantalum oxide (TaOₓ) and iridium oxide (IrOₓ). Iridium oxide can be particularly useful for intravascular stent applications because it exhibits good vascular compatibility.

As stated above, ceramic layers coating stent bodies can be formed to store and release drugs, i.e., therapeutic agents. Drugs can be loaded into the porous surface layer (e.g. ceramic layer 36) by a variety of suitable processes. In one example, one or more drugs are dissolved in water or an organic solvent, and a stent with ceramic coatings as described above is soaked in the solution for a period of time. The drug-loaded stent is subsequently dried off by evaporation, either at elevated temperatures or room temperature.

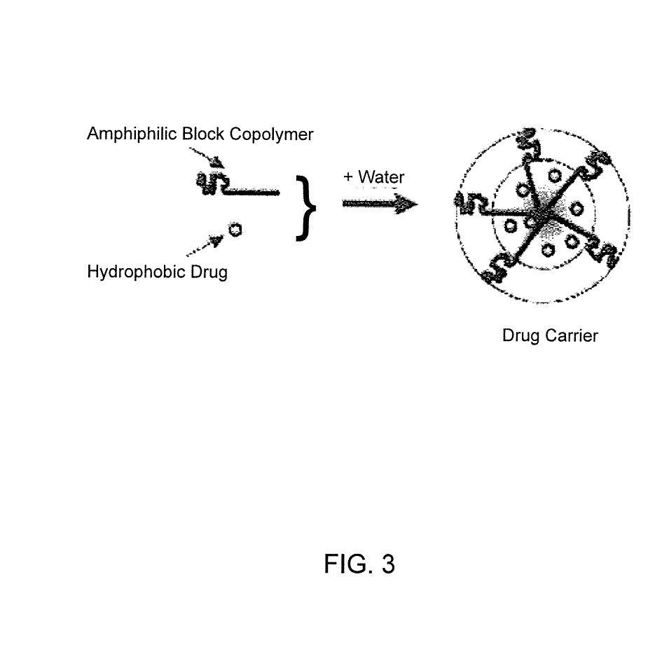

In another example, the drugs to be loaded into a stent are incorporated into the sol during the sol-gel process of making the ceramic coating layers. For example, drugs can be added to the ethanol solution of titanium alkoxide precursors, such as Ti(OiPr)₄ and polymers such as Pluronic or PEG during the sol-gel process. In one configuration, the drug molecules form complexes, such as micelles, with the components of the precursors, resulting in a more intimate association between the drug molecules and the ceramic layer, thereby forming a more robust drug eluting ceramic layer. For example, hydrophobic molecules of Paclitaxel, a chemotherapy drug, can form complexes with the amphiphilic block copolymers of Pluronic or PEG, as schematically illustrated in FIG. 3. In such applications, it is often desirable to have a sol with a low supercritical temperature so that the drying process does not drive off the drug molecules, which are typically of low molecular weight. Low supercritical temperatures can be achieved by proper choice of solvothermal processes, as discussed above.

In a further configuration, hybrid surface coating layers 32, 36 can be formed, with both polymeric and ceramic materials present in the layers. Such layers can be made by, for example, dissolving ceramic precursor, as discussed above, as well as polymer in the sol. The resultant surface coating layers inter-penetrating ceramic and polymeric networks. The presence of polymers in the surface layers provides at least two benefits in many applications. First, polymers can provide better bonding sites for drugs, which are typically organic, and compared to ceramic materials and thus a more robust drug reservoir. Second, the presence of a polymer network tends to make the surface coating layer more resilient, thereby enabling the surface coating layers to better accommodate the deformation of the stent during deployment.

It should be noted that, although the ceramic coating layers are disposed over abluminal or adluminal surfaces of stent bodies according to certain aspects of the present disclosure, steps of formation of ceramic coating layers need not be carried out while the stent body is in tubular form.

### III. Summary

Thus, ceramic coatings on stent bodies can be produced, with different surface morphologies coexisting in the same stent. Certain surface portions of the stent can be made relatively smooth to be more conducive to endothelial cell growth, thereby enhancing the incorporation of the stent into the host body tissue. Other surface portions of the stent can be made to be porous, for example, by etching the stent body portions upon which ceramic coatings are formed, to be more efficient in storing drugs. The sol-gel process for forming the ceramic coating results in strong bonding between the stent body and the ceramic coating, thereby enhancing the integrity of the coating and reducing the risk of breakage of the coating when the stent undergoes deformation during deployment.

The above specification, examples and data provide a complete description of the manufacture and use of the composition of the invention. Since many embodiments of the invention can be made without departing from the spirit and scope of the invention, the invention resides in the claims hereinafter appended.

We claim:

1. A method of making a stent, the method comprising:
coating at least a first surface portion of a metallic stent body material with a first layer comprising ceramic material, the stent body material having a nominal total surface area, the first surface comprising at least about 10% of the nominal total surface area and having a first average porosity; and
coating at least a second, etched surface portion of the metallic stent body material with second layer comprising ceramic material, the stent body material having a nominal total surface area, the first etched surface comprising at least about 10% of the nominal total surface area and having a second average porosity that is greater than the first average porosity by at least about 10%,
coating the first surface portion comprising coating the first portion with at least a first sol of ceramic precursor, and subsequently drying the first sol, and
coating the second surface portion comprising coating the second portion with at least a second sol of ceramic precursor, and subsequently drying the second sol.

2. The method of claim 1, further comprising creating the etched surface portion using chemical etching.

3. The method of claim 2, wherein chemical etching comprises masking the second surface portion with etching resist while etching the etched surface portions.

4. The method of claim 1, further comprising incorporating at least one drug into at least the second layer.

5. The method of claim 1, further comprising incorporating at least one drug into the sol of ceramic precursor.

6. The method of claim 1, further comprising providing a tubular stent body having at least one abluminal surface portion and at least one adluminal surface portion,
wherein coating the first surface portion comprises coating the at least one adluminal surface portion with the first layer comprising ceramic material; and
wherein coating the second surface portion comprises coating the at least one abluminal surface portion with second layer comprising ceramic material.

7. A method of making a stent, the method comprising:
coating at least a first surface portion of a metallic stent body material with a first layer comprising ceramic material; and
coating at least a second surface portion of the metallic stent body material with second layer comprising ceramic material, the second layer being more porous than the first layer by at least about 50%,
the stent body material having a nominal total surface area, the first surface portion of the metallic stent body comprising at least about 10% of the nominal total surface area, and the second surface portion of the metallic stent body comprising at least about 10% of the nominal total surface area.

8. The method of claim 7, further comprising making the second surface portions of the metallic stent rougher than the first surface portions.

9. The method of claim 8, wherein making the second surface portions rougher than the first surface portions comprises etching the second surface portions.

10. The method of claim 9, wherein etching comprises chemical etching.

11. The method of claim 11, wherein chemical etching comprises masking the first surface portions with etching resist.

12. The method of claim 7, wherein coating the first and second surface portions with the first and second layers, respectively, comprises coating the first and second surface portions with sol of ceramic precursor, and subsequently drying the ceramic precursor coated on the surface portions.

13. The method of claim 7, further comprising incorporating at least one drug into at least the second layer.

14. The method of claim 12, further comprising incorporating at least one drug into the sol of ceramic precursor.

15. The method of claim 8, further comprising providing a tubular stent body having abluminal surface portions and adluminal surface portions,
wherein coating first surface portions comprises coating the adluminal surface portions with the first layer comprising ceramic material; and
wherein coating second surface portions comprises coating the abluminal surface portions with second layer comprising ceramic material.

16. A method of treating a patient, the method comprising:
implanting at least one stent having a nominal total surface area into a patient's body,
wherein the stent comprising a first surface portion comprising at least 10% of the nominal total surface area on the at least one stent for promoting growth thereon of tissue of the patient's body and a second surface portion comprising at least 10% of the nominal total surface area, the second surface portion being more porous than the first surface portion by at least 50% for storing drug and for releasing the drug into the patient's body.

17. The method of claim 16, wherein implanting at least one stent into a patient's body comprises introducing the at least one stent into a luminal cavity of the patient's body; wherein providing first surface portion comprises providing adluminal surface portion on the stent for growth thereon of tissue of the patient; and wherein providing the second surface portion comprises providing abluminal surface portion that is more porous than the adluminal surface portion by at least 50% for storing the drug and for releasing the drug into the patient.

18. A stent made by the method of claim 1.

19. A stent made by the method of claim 7.

20. A stent, comprising:
a metallic stent body having a nominal total surface area and deformable from a collapsed state to an expanded state and having at least a first and a second surface portions, each comprising at least 10% of the nominal total surface area, the first surface portion being an etched portion and more porous than the second portion by at least 50%;
a first layer coating the first surface portion and comprising at least a ceramic material; and
a second layer coating the second surface portion and comprising at least a ceramic material.

21. The stent of claim 20, wherein the etched surface portion comprises a chemically etched surface portion.

22. The stent of claim 20, wherein the etched surface portion comprises a laser etched surface portion.

23. The stent of claim 20, wherein first and second layers are made by sol-gel process.

24. The stent of claim 20, wherein the second surface portion comprises at least a portion of an adluminal surface portion of the stent body; and the at least one etched surface portion comprises at least a portion of an abluminal surface portion of the stent body.

25. The stent of claim 20, wherein the second layer further comprises one or more drugs incorporated therein.

26. The stent of claim 25, wherein the second layer is made by sol-gel process, and wherein the one or more drugs are incorporated into the second layer during the sol-gel process.

27. The stent of claim 20, wherein at least a portion of the first layer is epitaxial with the stent body, and wherein at least a portion of the second layer is epitaxial with the stent body.

28. The stent of claim 20, wherein each of the first and second layers comprises titanium oxide, tantalum or iridium oxide.

29. The stent of claim 20, wherein each of the first and second layers further comprises polymer.

30. The method of claim 1, wherein each of the first surface portion has a first surface structure, and the second surface portion has a second surface structure, coating the first and second surface portions comprising substantially replicating the first surface structure by coating the first surface portion with at least a sol of ceramic precursor, and subsequently drying the ceramic precursor and substantially replicating the second surface structure by coating the second surface portion with at least a sol of ceramic precursor, and subsequently drying the ceramic precursor.

31. The method of claim 7, wherein each of the first surface portion has a first surface structure, and the second surface portion has a second surface structure, coating the first and second surface portions comprising substantially replicating the first surface structure by coating the first surface portion with at least a sol of ceramic precursor, and subsequently drying the ceramic precursor and substantially replicating the second surface structure by coating the second surface portion with at least a sol of ceramic precursor, and subsequently drying the ceramic precursor.

32. The method of claim 1, wherein drying the first and second sols comprises drying both at 200° C. or lower.

33. The method of claim 4, wherein drying the first and second sols comprise drying both at below 200° C. or lower.
